(19) United States
(12) Patent Application Publication
(10) Pub. No.: US 2025/0099490 A1
(43) Pub. Date: Mar. 27, 2025

(54) 25-HYDROXYVITAMIN D AS A FEED ADDITIVE FOR AQUATIC ANIMALS

(71) Applicant: DSM IP Assets B.V., Heerlen (NL)

(72) Inventors: Sebastien RIDER, Kaiseraugst (CH); Eduardo Yugo YAMASHITA, Kaiseraugst (CH)

(21) Appl. No.: 18/730,645

(22) PCT Filed: Jan. 23, 2023
(86) PCT No.: PCT/EP2023/051503

§ 371 Date: Jul. 19, 2024

(30) Foreign Application Priority Data
Jan. 21, 2022 (EP) 22152709.6

(51) Int. Cl. A61K 31/593; A23K 20/158; A23K 20/174; A23K 40/25; A23K 40/30; A23K 50/80; A61K 47/44

(52) U.S. Cl. CPC A61K 31/593; A23K 20/158; A23K 20/174; A23K 40/25; A23K 40/30; A23K 50/80; A61K 47/44

(57) ABSTRACT

The present invention relates to a composition for aquatic animals comprising as active ingredient 25-hydroxyvitamin D, preferably 25-hydroxy vitamin D3 (25-OH D3). The present invention further relates to the use of 25-hydroxyvitamin D, preferably 25-hydroxyvitamin D3 (25-OH D3), as feed additive for aquatic animals including fish and shrimp. More particular, this invention relates to the use of 25-hydroxyvitamin D, preferably 25-hydroxyvitamin D3 (25-OH D3), for improving feed utilization and growth and performance parameters of the animals, such as the feed conversion ratio (FOR). Immunity, protein efficiency ratio (PER), specific growth rate (SGR), final body weight, feed efficiency (FE), nutrient utilization, body protein content, and/or carcass yield and/or for increasing levels of Vitamin D, preferably Vitamin D3, in the blood of aquatic animals.

### BACKGROUND OF THE INVENTION

One important factor in aquaculture is the turnover rate. The turnover rate is determined by how fast the fish grow to a harvestable size.

As an example, it takes from 12 to 18 months to raise Atlantic salmon from smolt (the physiological stage when the Atlantic salmon can first be transferred from fresh water to sea water) to harvestable size. A fast turnover has several positive results. First, it helps cash flow. Second, it improves risk management.

The turnover rate may e.g. be affected by growth, health and performance parameters, such as the Feed Conversion Rate (FCR). It therefore remains a constant need in aquaculture industry to improve such parameters.

### SUMMARY OF THE INVENTION

The inventors of the present application surprisingly found that 25-hydroxyvitamin D, preferably 25-hydroxy vitamin D3 (25-OH D3), has a great potential for use in fish feed, e.g. for improving feed utilization and growth and performance parameters of the animals, such as the feed conversion ratio (FCR), Immunity, protein efficiency ratio (PER), specific growth rate (SGR), final body weight, feed efficiency, nutrient utilization, body protein content and/or carcass yield. Especially surprising, it was found that 25-hydroxyvitamin D, preferably 25-hydroxy vitamin D3 (25-OH D3), improved carcass yields at the expense of peritoneal fat surrounding the viscera. Further surprisingly, it was found that 25-hydroxyvitamin D, preferably 25-hydroxy vitamin D3 (25-OH D3), increases levels of Vitamin D, preferably Vitamin D3, in the blood of aquatic animals, even when they were Vitamin D3 replete.

Therefore, in one aspect, the present invention relates to a composition comprising as active ingredient 25-hydroxyvitamin D, preferably 25-hydroxy vitamin D3 (25-OH D3), wherein the composition is selected from the group consisting of a feed additive, a feed premix or aquaculture feed, wherein the and the concentration of the active ingredient in the final feed added to the animal is in the range from 50 μg to 1500 μg per kg feed, preferably 100 μg to 1500 μg per kg feed, preferably 150 μg to 1500 μg per kg feed, more preferably 150 μg to 1250 μg per kg feed, more preferably 150 μg to 1000 μg per kg feed.

In a further aspect, the present invention relates to a method of preparing a feed pellet comprising 25-hydroxyvitamin D, preferably 25-hydroxy vitamin D3 (25-OH D3).

In one embodiment, said method of preparing a feed pellet comprising 25-hydroxyvitamin D, preferably 25-hydroxy vitamin D3 (25-OH D3), comprising the steps of:
i) combining feed ingredients ii) forming a fish feed pellets comprising said feed ingredients, (iii) obtaining a feed pellet, (iv) coating said pellet with or in an oil comprising 25-hydroxyvitamin D, preferably 25-hydroxy vitamin D3 (25-OH D3), and (v) obtaining a feed pellet comprising 25-hydroxyvitamin D, preferably 25-hydroxy vitamin D3 (25-OH D3), in a concentration of between 50 μg to 1500 μg per kg feed, preferably 100 μg to 1500 μg per kg feed, preferably 150 μg to 1500 μg per kg feed, more preferably 150 μg to 1250 μg per kg feed, more preferably 150 μg to 1000 μg per kg feed.

In another embodiment, said method of preparing a feed pellet comprising 25-hydroxyvitamin D, preferably 25-hydroxy vitamin D3 (25-OH D3), comprising the steps of:
i) combining feed ingredients, including 25-hydroxyvitamin D, preferably 25-hydroxy vitamin D3 (25-OH D3) ii) forming a fish feed pellets comprising said feed ingredients, (iii) obtaining a feed pellet, and optionally the further steps of (iv) coating said pellet with or in an oil, and (v) obtaining a feed pellet comprising 25-hydroxyvitamin D, preferably 25-hydroxy vitamin D3 (25-OH D3), in a concentration of between 50 μg to 1500 μg per kg feed, preferably 100 μg to 1500 μg per kg feed, preferably 150 μg to 1500 μg per kg feed, more preferably 150 μg to 1250 μg per kg feed, more preferably 150 μg to 1000 μg per kg feed.

In a third aspect, the present invention pertains to the use of 25-hydroxyvitamin D, preferably 25-hydroxy vitamin D3 (25-OH D3), for
improving feed conversion ratio in aquatic animals,
improving the protein efficiency ratio (PER) in aquatic animals,
improving the growth performance (specific growth rate SGR) in aquatic animals,
improving final body weight in aquatic animals
improving carcass yield in aquatic animals, and/or
for increasing levels of Vitamin D, preferably Vitamin D3, in the blood of aquatic animals.

In a fourth aspect, the invention relates to methods for
improving feed conversion ratio in aquatic animals,
improving the protein efficiency ratio (PER) in aquatic animals,
improving the growth performance (specific growth rate SGR) in aquatic animals,
improving final body weight in aquatic animals
improving carcass yield in aquatic animals, and/or
for increasing levels of Vitamin D, preferably Vitamin D3, in the blood of aquatic animals,
   said methods comprise feeding to the aquatic animal a feed comprising 25-hydroxyvitamin D, preferably 25-hydroxy vitamin D3 (25-OH D3).

In a fith aspect, the present invention pertains to a composition comprising 25-hydroxyvitamin D, preferably 25-hydroxy vitamin D3 (25-OH D3), for use in
improving feed conversion ratio in aquatic animals,
improving the protein efficiency ratio (PER) in aquatic animals,
improving the growth performance (specific growth rate SGR) in aquatic animals,
improving final body weight in aquatic animals
improving carcass yield in aquatic animals, and/or
for increasing levels of Vitamin D, preferably Vitamin D3, in the blood of aquatic animals.

In a further aspect, the present invention pertains to a feed additive composition comprising 25-hydroxyvitamin D, preferably 25-hydroxy vitamin D3 (25-OH D3), in the form of a powder, optionally embedded in an oil carrier.

In a further aspect, the present invention pertains to a premix composition or aquaculture feed additive comprising 25-hydroxyvitamin D, preferably 25-hydroxy vitamin D3 (25-OH D3), and at least one additional component selected from the group consisting of fat-soluble vitamins, water soluble vitamins, carotenoids, polyunsaturated fatty acids, trace minerals, probiotics, prebiotics and macro minerals.

### BRIEF DESCRIPTION OF THE FIGURES

FIG. 1. Effect of 25-OH-D₃ on growth performance in rainbow trout; FIG. 1 shows the increase in specific growth rate (SGR) relative to the control (0%) for each two-week period in fish fed control diet or fish supplemented 80, 800, or 8000 μg/kg 25-OH-D₃ (as fed basis). An increased growth rate relative to the control in fish fed diets with 25-OH-D₃ was apparent from 31 days onwards.

FIG. 2. Effect of 25-OH-D₃ on feed conversion in rainbow trout; FIG. 2 shows the rapidly improved feed conversion ratio (FCR) relative to the control (0%) from 15 days onwards in fish fed the same diets containing supplemental 80, 800, or 8000 μg/kg 25-OH-D₃.

FIG. 3. Effect of dietary 25-OH-D₃ on 25-OH-D₃ in blood plasma of rainbow trout;

FIG. 3 show the linear increase of blood plasma 25-OH-D₃ in response to increasing dietary 25-OH-D₃ confirming the bioavailability of 25-OH-D₃ from Hy·D supplemented as 80, 800, or 8000 μg/kg to a standard trout feed.

FIG. 4. Effect of dietary 25-OH-D₃ on 25-OH-D₃ in muscle of rainbow trout; FIG. 4 show the linear increase of muscle 25-OH-D₃ in response to increasing dietary 25-OH-D₃ confirming the bioavailability of 25-OH-D₃ from Hy·D supplemented as 80, 800, or 8000 μg/kg to a standard trout feed.

FIG. 5. Effect of dietary 25-OH-D₃ on 1,25-OH-D₃ in blood plasma of rainbow trout; FIG. 5 shows the increased plasma levels of the active D₃ metabolite, 1,25-OH-D₃ in fish fed 25-OH-D₃ from Hy·D, confirming an increased vitamin D₃ status with 80, 800, or 8000 μg/kg dietary 25-OH-D₃ from Hy·D.

FIG. 6. Effect of dietary 25-OH-D₃ on D₃ in blood plasma of rainbow trout; FIG. 6 shows 80, 800, or 8000 μg/kg Hy·D also results in in elevated plasma levels of D₃ compared to the control.

FIG. 7. Effect of 25-OH-D₃ on growth performance in Atlantic salmon; FIG. 7 shows the increase in mean final body weight±standard deviation (SD) in fish supplemented 100, 200, or 400 μg/kg 25-OH-D₃ (as fed basis). An increased growth in fish fed diets with 25-OH-D₃ relative to the control was apparent for all groups supplemented 25-OH-D₃.

FIG. 8. Effect of 25-OH-D₃ on feed conversion in Atlantic salmon; FIG. 8 shows the improved mean±SD feed conversion (FCR) in the same fish fed diets containing supplemented 100 ug, 200 ug, 400 ug/kg 25-OH-D₃.

FIG. 9. Effect of 25-OH-D₃ on protein efficiency ratio (PER) in Atlantic salmon;

FIG. 9 shows the improved mean±SD protein utilization (PER) in the same fish fed diets containing supplemented 100 μg, 200 μg, 400 ug/kg 25-OH-D₃.

FIG. 10. Effect of 25-OH-D₃ on viscera in Atlantic salmon; FIG. 10 shows the decrease in mean viscerosomatic index (VSI)+standard deviation (SD) in fish supplemented 100, 200, or 400 μg/kg 25-OH-D₃ (as fed basis). A decreased VSI in fish fed diets with 25-OH-D₃ relative to the control was apparent for all groups supplemented 25-OH-D₃.

FIG. 11. Effect of 25-OH-D₃ on carcass yields n Atlantic salmon; FIG. 11 shows the increased mean±SD percentage carcass in the same fish fed diets containing supplemented 100 μg, 200 μg, 400 ug/kg 25-OH-D₃

FIG. 12. Effect of 25-OH-D₃ on visceral fat in Atlantic salmon; FIG. 12 shows the mean±SD percentage fat in the viscera of the same fish fed diets containing supplemented 100 μg, 200 μg, 400 ug/kg 25-OH-D₃.

### DEFINITIONS

Aquatic Animal: The term “aquatic animal” refers to crustaceans including but not limited to shrimps and prawns and fish including but not limited to amberjack, arapaima, barb, bass, bluefish, bocachico, bream, bullhead, cachama, carp, catfish, catla, chanos, char, cichlid, cobia, cod, crappie, dorada, drum, eel, goby, goldfish, gourami, grouper, guapote, halibut, java, labeo, lai, loach, mackerel, milkfish, mojarra, mudfish, mullet, paco, pearlspot, pejerrey, perch, pike, pompano, roach, salmon, sampa, sauger, sea bass, seabream, shiner, sleeper, snakehead, snapper, snook, sole, spinefoot, sturgeon, sunfish, sweetfish, tench, terror, tilapia, trout, tuna, turbot, vendace, walleye and whitefish.

Basal diet: “Basal diet” means that the feed used supplies the poultry with sufficient vitamins and minerals so that the poultry are vitamin and mineral replete.

Feed Additive: The term feed additive according to the invention refers to a formulation comprising 25-hydroxyvitamin D, preferably 25-hydroxy vitamin D3 (25-OH D3), as active ingredient intended for intake by the fish.

Feed Premix: The incorporation of the composition of feed additives as exemplified herein above to animal feeds, for example fish feeds, is in practice carried out using a concentrate or a premix. A premix designates a preferably uniform mixture of one or more microingredients with diluent and/or carrier. Premixes are used to facilitate uniform dispersion of micro-ingredients in a larger mix. A premix according to the invention can be added to feed ingredients as solids (for example as water soluble powder) or liquids.

Feed or Aquaculture feed: The term “Feed” or “Aquaculture feed” or “aquatic feed” refers to any compound, preparation, or mixture suitable for, or intended for intake by aquatic animals and decapod crustacean. An animal feed for aquatic animals typically comprises high protein and energy concentrations, such as fish meal, molasses, oligosaccharide concentrates as well as vitamins, minerals, enzymes, direct fed microbial, amino acids and/or other feed ingredients (such as in a premix). Aquaculture feed refers to a manufactured or artificial diet (i.e., formulated feed) to supplement or to replace natural feed, which is most commonly produced in form of flakes or pellets. Preferred embodiment of feed pellets are characterized by a pellet size (diameter) in a range from 0.5 to 16 mm pellet size (diameter) roughly and a protein-content from 20% to 65% w/w.

Typically, a decapod crustacean feed may be in the form of flakes or pellets, for example extruded pellets. In the present context the term “decapod crustacean feed” may e.g. be a shrimp or prawn feed.

Feed efficiency: The term “feed efficiency” means the amount of weight gain per unit of feed when the animal is fed ad-libitum or a specified amount of food during a period of time. By “increased feed efficiency” it is meant that the use of a feed additive composition according the present invention in feed results in an increased weight gain per unit of feed intake compared with an animal fed without said feed additive composition being present.

Final body weight: The term “final body weight” means the eight of an animal after a set period of time. By “increased final body weight” it is meant that the use of a feed additive composition according the present invention in feed results in an increased body weight of an animal compared with an animal fed without said feed additive composition being present.

Feed Conversion Ratio (FCR): FCR is a measure of an animal's efficiency in converting feed mass into increases of the desired output. Animals raised for meat—such as swine, poultry and fish—the output is the mass gained by the animal. Specifically, FCR is calculated as feed intake divided by weight gain, all over a specified period. Improvement in FCR means reduction of the FCR value. An FCR improvement of 2% means that the FCR was reduced by 2%.

protein efficiency ratio (PER): The term protein efficiency ratio (PER) according to the invention refers to the weight gain of a test subject divided by its intake of a particular food protein during the test period. Therefore, weight gain/protein consumed, wherein protein consumed=feed consumed/% crude protein in feed. By “improving protein efficiency ratio (PER)” it is meant that the use of a composition according the present invention in feed results in an increased weight gain per unit of feed intake compared with an animal fed without said feed additive composition being present.

carcass yield: The term carcass means the body of an animal, that has been slaughtered for food, with the head, limbs, skinned or scalded and entrails removed. The term carcass yield is determined on the basis of the carcass and of body weight of the animal before slaughtering according to the formula [dressed carcass weight/live weight]×100. By “improving carcass yield” it is meant that the use of a composition according the present invention in feed results in an higher ratio of dressed carcass weight to live weight (in %) compared with an animal fed without said feed additive composition being present.

Mash: The term “Mash” or “Feed Mash” or “Mash Feed” according to the invention refers to the simplest solid feed form that can be manufactured. It consists of grinding and mixing all raw materials into the correct proportions to meet nutritional requirements of the animal.

Specific Growth Rate (SGR): The term Specific Growth Rate (SGR) according to the invention refers to the daily increase in bodyweight (in %). In the context of the present invention, growth performance means specific growth rate (SGR). By “improving growth performance” or “improving specific growth rate (SGR)” it is meant that the use of a composition according the present invention in feed results in an higher daily increase in bodyweight (in %) compared with an animal fed without said feed additive composition being present.

25-OH D: “25-OH D” or “25-hydroxyvitamin D” refers to any form of 25-hydroxyvitamin D (i.e. either 25-OH D2 or 25-OH D3, or mixes thereof). 25-OH D3 specifically refers to 25-hydroxyvitamin D3 (also known as calcifediol, calcidiol, 25-hydroxycholecalciferol or 25-hydroxyvitamin D₃); 25-OH D2 specifically refers to 25-hydroxyvitamin D2. In a preferred embodiment, 25-hydroxyvitamin D is 25-hydroxyvitamin D3.

ROVIMIX® Hy·DR1.25%: 25-hydroxyvitamin D3 is commercially available for example in the feed additive ROVIMIX® Hy·DR1.25% (also referred to as Hy·D). ROVIMIX® Hy·DR1.25% is a beige to brown fine powder that contains 12.5 g/kg 25-OH-D3 as the active substance. One kilogram of ROVIMIX® Hy·DR1.25% also contains 37.5 g antioxidant (authorized for feed use), 25 g sodium ascorbate, 50 g vegetable oil, 715 g modified food starch, 150 g maltodextrin and 10 g silicon dioxide.

Visceral somatic index: The term Visceral somatic index (VSI) is calculated as VSI=(Total weigh of all viscera/total body weight of the fish prior to removal of the viscera)*100.

Visceral fat: Visceral fat is a type of body fat that is stored within the abdominal cavity. It is located near several vital organs, including the liver, stomach, and intestines.

Vitamin D: “Vitamin D” means either Vitamin D2, Vitamin D3 or a combination. Vitamin D3 is preferred.

1,25 dihydroxycholecalciferol (1,25-OH-D3): 1,25 dihydroxycholecalciferol (1,25-OH-D3) or calcitriol is the active form of vitamin D, normally made in the kidney.

### DETAILED DESCRIPTION OF THE INVENTION

The present invention relates to the use of 25-hydroxyvitamin D, preferably 25-hydroxy vitamin D3 (25-OH D3), as feed additive for aquatic animals including fish and decapod crustaceans.

More particular, this invention relates to the use of 25-hydroxyvitamin D, preferably 25-hydroxy vitamin D3 (25-OH D3), for
improving feed conversion ratio in aquatic animals,
improving the protein efficiency ratio (PER) in aquatic animals,
improving the growth performance (specific growth rate SGR) in aquatic animals,
improving final body weight in aquatic animals
improving carcass yield in aquatic animals, and/or
for increasing levels of Vitamin D, preferably Vitamin D3, in the blood of aquatic animals.

Furthermore, the present invention relates to a novel aquatic feed composition comprising as active ingredient 25-hydroxyvitamin D, preferably 25-hydroxy vitamin D3 (25-OH D3), wherein the composition is selected from the group consisting of a feed additive, a feed premix or aquaculture feed.

In a first particular embodiment, the invention relates to methods for using 25-hydroxyvitamin D, preferably 25-hydroxy vitamin D3 (25-OH D3), for
improving feed conversion ratio in aquatic animals,
improving the protein efficiency ratio (PER) in aquatic animals,
improving the growth performance (specific growth rate SGR) in aquatic animals,
improving final body weight in aquatic animals
improving carcass yield in aquatic animals, and/or
for increasing levels of Vitamin D, preferably Vitamin D3, in the blood of aquatic animals.

In preferred embodiments, the 25-hydroxyvitamin D, preferably 25-hydroxy vitamin D3 (25-OH D3), is added to a basal diet which contains all the necessary ingredients for complete fish nutrition, i.e. the combination provides a supra-physiological amount of the components. Thus, this can be distinguished from prior use of the active ingredient which is provided in order to merely meet nutritional requirements so that the fish is not vitamin or nutrient deficient.

The FCR may be determined on the basis of a growth trial comprising a first treatment in which a mixture of at least two compounds according to the invention is added to the animal feed in a suitable concentration per kg feed, and a second treatment (control) with no addition of the compound(s) to the animal feed.

As it is generally known, an improved FCR is lower than the control FCR. In particular embodiments, the FCR is improved (i.e., reduced) as compared to the control by at least 1.0%, preferably at least 1.5%, 1.6%, 1.7%, 1.8%, 1.9%, 2.0%, 2.1%, 2.2%, 2.3%, 2.4% or at least 2.5%.

In one aspect of this invention the 25-hydroxyvitamin D, preferably 25-hydroxy vitamin D3 (25-OH D3), is given to animals which are vitamin replete rather than vitamin deficient. The vitamin replete status is preferably due to the use of a basal feed which supplies at least the minimum amount of vitamins and minerals for the animals. The combination of this invention is thus preferably used in addition to the basal diet.

In a particular embodiment, the invention relates to a composition comprising as active ingredient 25-hydroxyvitamin D, preferably 25-hydroxy vitamin D3 (25-OH D3), for use in
improving feed conversion ratio in aquatic animals,
improving the protein efficiency ratio (PER) in aquatic animals,
improving the growth performance (specific growth rate SGR) in aquatic animals,
improving final body weight in aquatic animals
improving carcass yield in aquatic animals, and/or
for increasing levels of Vitamin D, preferably Vitamin D3, in the blood of aquatic animals.

A feed additive composition according to the invention can be made as described in example 3. The active ingredient of such a composition can be 25-hydroxyvitamin D, preferably 25-hydroxy vitamin D3 (25-OH D3). The 25-hydroxyvitamin D, preferably 25-hydroxy vitamin D3 (25-OH D3), may be embedded in an oil carrier.

Said oil carrier can be fish oil, microbial oil and/or one or more vegetable oil(s). The vegetable oil can be selected from the group consisting of rape seed oil and soy oil. An example of a microbial oil according to the invention is an oil from Schizochytrium. Preferably, the oil is a source of eicosapentaenoic acid (“EPA”) and/or docosahexaenoic acid (“DHA”). “Eicosapentaenoic acid” [“EPA”] is the common name for eis-5, 8, 11,14, 17-eicosapentaenoic acid. This fatty acid is a 20:5 omega-3 fatty acid. “Docosahexaenoic acid” [“DHA”] is the common name for eis-4, 7, 10, 13, 16, 19-docosahexaenoic acid. This fatty acid is a 22:6 omega-3 fatty acid.

The incorporation of the feed additive composition containing the 25-hydroxyvitamin D, preferably 25-hydroxy vitamin D3 (25-OH D3), into fish feed may be performed as described in example 1 and 2. The final concentration of 25-hydroxyvitamin D, preferably 25-hydroxy vitamin D3 (25-OH D3), in the feed is determined by HPLC according to standard methods.

The incorporation of the feed additive composition containing the 25-hydroxyvitamin D, preferably 25-hydroxy vitamin D3 (25-OH D3), into fish feed may alternatively be carried out by preparing a premix of the 25-hydroxyvitamin D, preferably 25-hydroxy vitamin D3 (25-OH D3), and other suitable additives. Such a premix may comprise 0-40% by weight of other conventional additives, such as flavorings, and 50-98% by weight of any conventional absorbing support.

The support may contain, for example, 40-50% by weight of wood fibers, 8-10% by weight of stearin, 4-5% by weight of curcuma powder, 4-5% by weight of rosemary powder, 22-28% by weight of limestone, 1-3% by weight of a gum, such as gum Arabic, 5-50% by weight of sugar and/or starch and 5-15% by weight of water.

The premix may also contain vitamins, as for example vitamin E, mineral salts and other feed additive ingredients, as for example yeast extracts containing nucleotides, glucan and other gut microflora modulators such as pro- and/or prebiotics and then finally added to the feed in such quantities that the feed comprises 10-5000 ppm, preferably 100-1000 ppm, 150-1000 ppm, 500-1000, 500-750 or 100-500 ppm of 25-hydroxyvitamin D, preferably 25-hydroxy vitamin D3 (25-OH D3), on.

Further, optional, feed-additive ingredients which can be added to the premix are coloring agents, e.g. carotenoids such as beta-carotene, astaxanthin, and lutein; aroma compounds; stabilisers; antimicrobial peptides; polyunsaturated fatty acids; and/or at least one enzyme selected from amongst phytase (EC 3.1.3.8 or 3.1.3.26); xylanase (EC 3.2.1.8); galactanase (EC 3.2.1.89); alpha-galactosidase (EC 3.2.1.22); protease (EC 3.4.), phospholipase A1 (EC 3.1.1.32); phospholipase A2 (EC 3.1.1.4); lysophospholipase (EC 3.1.1.5); phospholipase C (EC 3.1.4.3); phospholipase D (EC 3.1.4.4); amylase such as, for example, alpha-amylase (EC 3.2.1.1); and/or beta-glucanase (EC 3.2.1.4 or EC 3.2.1.6).

Examples of polyunsaturated fatty acids are C18, C20 and C22 polyunsaturated fatty acids, such as arachidonic acid, docosohexaenoic acid, eicosapentaenoic acid and gamma-linoleic acid. Fish oil, microbial oil and/or one or more vegetable oil(s) are sources of these fatty acids. The vegetable oil can be selected from the group consisting of rape seed oil and soy oil. An example of a microbial oil according to the invention is an oil from Schizochytrium.

Another preferred premix composition according to the invention comprises as active ingredient 25-hydroxyvitamin D, preferably 25-hydroxy vitamin D3 (25-OH D3), and a coating oil in which the active ingredient is dissolved or suspended.

Said coating oil can be fish oil, microbial oil and/or one or more vegetable oil(s). The vegetable oil can be selected from the group consisting of rape seed oil and soy oil. An example of a microbial oil according to the invention is an oil from Schizochytrium. Preferably, the oil is a source of eicosapentaenoic acid (“EPA”) and/or docosahexaenoic acid (“DHA”).

In another embodiment, the invention relates to a feed or fish feed composition for aquatic animals.

The term “feed” or “fish feed” or “aquatic feed” as used herein includes a fish feed composition according to the invention and components as described above. Typically, fish feed includes fish meal as a component. Suitably, fish feed is in the form of flakes or pellets, for example extruded pellets.

In one embodiment the feed comprises from 50 μg 25-hydroxyvitamin D, preferably 25-hydroxy vitamin D3 (25-OH D3), per kg feed, such as in the range from 50 μg to 1500 μg mg 25-hydroxyvitamin D, preferably 25-hydroxy vitamin D3 (25-OH D3), per kg feed, e.g. in the range from 150 μg to 1250 μg 25-hydroxyvitamin D, preferably 25-hydroxyvitamin D3 (25-OH D3), per kg feed, such as in the range from 150 μg to 1000 μg 25-hydroxyvitamin D, preferably 25-hydroxyvitamin D3 (25-OH D3), per kg feed.

In one embodiment the feed comprises more than 100 μg 25-hydroxyvitamin D, preferably 25-hydroxyvitamin D3 (25-OH D3), per kg feed, preferably from 150, 250, 300, 350, 400, 450, 500 μg per kg feed.

In one embodiment the feed comprises up to 1500 μg 25-hydroxyvitamin D, preferably 25-hydroxyvitamin D3 (25-OH D3), per kg feed, preferably up to 1400, 1300, 1250, 1200, 1100, 1000, 900, 850, 800 μg per kg feed.

The fish feed as described herein comprises a proximate composition of 20-60 wt.-% protein, and 1-45 wt.-% moisture and lipid.

In one embodiment the aquatic animal feed is a fish feed or a decapod crustacean feed.

In some specific examples, the aquatic feed comprises one or more of sources of:
protein, carbohydrate and lipid (for example, fish meal, fish oil, blood meal, feather meal, poultry meal, chicken meal and/or other types of meal produced from other slaughterhouse waste),
animal fat (for example poultry oil),
vegetable meal (e.g. soya meal, lupin meal, pea meal, bean meal, rape meal and/or sunflower meal),
vegetable oil (e.g. rapeseed oil, soya oil and/or camelina oil),
gluten (e.g. wheat gluten or corn gluten) and
added amino acids (e.g. lysine)
ash
moisture such as e.g. water.

Thus, in one embodiment the aquatic animal feed of the present invention may comprise ingredients selected from the group consisting of a carbohydrate source, a protein source, a lipid source, ash, water and any combinations thereof.

Typically, the protein source may constitute from 20-60% (w/w) of the composition and/or fish feed, such as from 26-54% (w/w), e.g. from 27-53% (w/w), such as from 28-52% (w/w), e.g. from 27-51% (w/w), such as from 28-50% (w/w), e.g. from 29-49% (w/w), such as from 30-48% (w/w), e.g. from 31-47% (w/w), such as from 32-46% (w/w), e.g. from 33-45% (w/w), such as from 34-44% (w/w), e.g. from 35-43% (w/w), such as from 36-42% (w/w), such as from 37-41% (w/w), e.g. from 38-40% (w/w), such as from 39-40% (w/w), e.g. from 20-40% (w/w), such as from 25-60% (w/w), preferably in the range from 30-55% w/w.

The carbohydrate source may constitute from 10-25% (w/w) of the composition and/or fish feed, such as from 11-24% (w/w), e.g. from 12-23% (w/w), such as from 13-24% (w/w), e.g. from 14-23% (w/w), such as from 15-24% (w/w), e.g. from 16-23% (w/w), such as from 17-22% (w/w), e.g. from 18-21% (w/w), such as from 19-20% (w/w), preferably in the range from 10-15% (w/w).

The lipid source may constitute from 15-40% (w/w) of the composition and/or fish feed, such as from 16-39% (w/w), e.g. from 17-38% (w/w), e.g. 18-37% (w/w), such as from 19-36% (w/w), e.g. from 20-35% (w/w), such as from 21-36% (w/w), e.g. from 22-35% (w/w), such as from 23-36% (w/w), e.g. from 24-35% (w/w), such as from 25-34% (w/w), e.g. from 26-33%, such as from 27-32% (w/w), e.g. from 28-33%, such as from 29-32% (w/w), e.g. from 30-31% preferably in the range from 25-40% (w/w).

In a further embodiment, the invention relates to a method of providing an extruded feed pellet.

Feed pellets (such as aquatic feed pellets) according to the present invention may be extruded pellets. The extruded feed pellets may be produced by the method of the present invention. The 25-hydroxyvitamin D, preferably 25-hydroxyvitamin D3 (25-OH D3), may be added to the extruded feed pellets pre or post extrusion. Pre extrusion, the 25-hydroxyvitamin D, preferably 25-hydroxyvitamin D3 (25-OH D3), is added to the mash. Post extrusion, the 25-hydroxyvitamin D, preferably 25-hydroxyvitamin D3 (25-OH D3), is added in a coating onto the extruded feed pellets.

In preferred examples the final feed pellets comprises 1 to 40%, for example 12 to 45% coating oil according to the invention.

It may be contemplated that the extruded feed pellet(s) has a DORIS value in the range from 75-100%. The DORIS value is measured on a DORIS tester (Durability on a Realistic Test) (Akvasmart, AKVA group ASA, Bryne, Norway). The DORIS tester is designed to mimic the pellet degradation during pneumatic feeding system. The result is between 0% and 100%, and corresponds to the mass fraction (m/m) of whole pellets after DORIS exposure relative to the initial sample mass.

It may also be contemplated that the extruded feed pellets of the present invention has a pellet hardness in the range from 20-100N. The pellet hardness is determinded on a pellet strength texture analyzer from Stable Micro Systems Ltd, Godalming, More specifically a TA-XT plus Texture Analyzer (TA) from Stable Micro Systems mounted with a cylindrical probe (P/40) was used to determine pellet hardness.

The feed can be fed to all types of fish, including cold-water fish and shrimp. Some examples are turbot, halibut, yellow tail salmon, trout, bream, bass and tuna. The feed is particularly suitable for feeding salmonids, including Atlantic salmon (Salmo salar), other salmon species and trout, and non-salmonids such as cod, sea bass, sea bream and eel. It is suitable for feeding salmon, trout, bream and/or bass in the fresh water (FW) phase and the in the sea water (SW) phase and in the period after hatching and until slaughter and in all stages, such as fry, fingerlings, parr, smolts and adult fish.

The composition of the present invention (i.e. a feed, a feed additive, a premix and an oil) may be particularly suitable for aquatic animals and for a variety of aquatic animal species.

In a particular preferred embodiment of the present invention, the aquatic animal is a fish or a decapod crustacean.

The fish may be any kind of fish such as but not limited to a fish selected from the group consisting of salmon, trout, sea bream, sea bass, cod, eel, turbot, halibut, yellow tail, tuna, carp, tilapia and catfish. In a particular preferred embodiment, the fish is selected from the group consisting of salmon, trout, sea bream and sea bass.

The salmon may be of the family Salmonidae and of the subfamily of Salmoninae. In one embodiment the salmon is selected from the group consisting of the genus Salmo, Oncorhynchus and Salvenis. In a further embodiment the genus Salmo is selected from the group consisting of Atlantic salmon (Salmo salar) and Brown trout (Salmo trutta). In yet an embodiment the genus Oncorhynchus is selected from the group consisting of Chinook salmon (Oncorhynchus tshawytsch), Rainbow trout (Oncorhynchus mykiss), Sockeye salmon (Oncorhynchus nerka) and Coho salmon (Oncorhynchus kisutch). In a further embodiment the genus Salvenis is selected from the group consisting of Arctic charr (Salvelinus alpinus), Brook trout (Salvelinus fontinalis) and Lake trout (Salvelinus namaycush).

The sea bream may be gilt-head sea bream (Sparus aurata) wheras the sea bass may be European bass (Dicentrarchus labrax)

In a preferred embodiment of the present invention the decapod crustacean may be shrimp or prawn.

The shrimp may be selected from the group consisting of Pacific white shrimp (Penaeus vannamei or Litopenaeus vannamei), Whiteleg shrimp (Penaeus vannamei or Litopenaeus vannamei), Black tiger shrimp (Penaeus monodon), Kuruma shrimp (Penaeus japonicas or Marsupenaeus japonicas), Western blue shrimp (Penaeus stylirostris or Litopenaeus stylirostris), blue shrimp (Penaeus stylirostris or Litopenaeus stylirostris), Chinese white shrimp (Penaeus chinensis or Fenneropenaeus chinensis), Oriental shrimp (Penaeus chinensis or Fenneropenaeus chinensis), Indian white shrimp (Penaeus indicus or Fenneropenaeus indicus), Banana shrimp (Penaeus merguiensis or Fenneropenaeus merguiensis), Akiami paste shrimp (Metapenaeus spp.), yellowleg shrimp (Penaeus californiensis or Farfantepenaeus californiensis), brown shrimp (Penaeus californiensis or Farfantepenaeus californiensis), São Paulo shrimp (Penaeus paulensis or Farfantepenaeus paulensis), Carpas shrimp (Penaeus paulensis or Farfantepenaeus paulensis), redspotted shrimp (Penaeus brasiliensis or Farfantepenaeus brasiliensis), spotted pink shrimp (Penaeus brasiliensis or Farfantepenaeus brasiliensis) and southern white shrimp (Penaeus schmitti).

The prawn may be selected from the group consisting of King prawn (Penaeus vannamei or Litopenaeus vannamei), Giant tiger prawn (Penaeus monodon), Giant Freshwater Prawn (Macrobrachium rosenbergii), Giant river prawn (Macrobrachium rosenbergii), Malaysian prawn (Macrobrachium rosenbergii), Kuruma prawn (Penaeus japonicas or Marsupenaeus japonicas), Fleshy prawn (Penaeus chinensis or Fenneropenaeus chinensis), Indian prawn (Penaeus indicus or Fenneropenaeus indicus), Banana prawn (Penaeus merguiensis or Fenneropenaeus merguiensis), Oriental river prawn (Macrobrachium nipponense) and Monsoon river prawn (Macrobrachium malcolmsonii).

The composition of the present invention may be administered (i.e. fed) to the aquatic animal in the period after hatching and until slaughter. This also means that the composition may be administered (i.e. fed) the aquatic animal in fresh water (FW) or in sea water (SW) dependent on the life stage of the aquatic animal.

In respect of fish the composition of the present invention may be administered (i.e. fed) to the fish during all life stages and thus, the fish may be selected from the group consisting of larvae, fry, fingerlings, parr, smolts and adult fish.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Embodiments of the invention can be summarized as follows:
1. A composition comprising as an active ingredient 25-hydroxyvitamin D, wherein the composition is intended for intake by aquatic animals and wherein and the concentration of the active ingredient in the final feed added to the animal is in the range from 50 μg to 1500 μg per kg feed, preferably 100 μg to 1500 μg per kg feed, preferably 150 μg to 1500 μg per kg feed, more preferably 150 μg to 1250 μg per kg feed, more preferably 150 μg to 1000 μg per kg feed.
2. The composition according to claim 1, wherein the 25-hydroxyvitamin D is 25-hydroxy vitamin D3 (25-OH D3).
3. The composition according to claim 1 or 2, wherein the active ingredient is embedded in an oil carrier.
4. The composition according to claim 3, wherein the oil carrier is a coating oil.
5. The composition according to any of claims 1 to 3, wherein said composition is a feed premix, wherein said premix comprises in addition to the active ingredient at least one additional component selected from the group consisting of fat-soluble vitamins, water soluble vitamins, trace minerals, carotenoids, polyunsaturated fatty acids, probiotics, prebiotics and macro minerals.
6. The composition according to any of claims 1 to 3, wherein the composition is an aquaculture feed and wherein the feed comprises at least one additional component, wherein
a. the additional component is 25-55% (w/w) of a protein source, or
b. the additional component is 10-25% (w/w) of a carbohydrate source, or
c. the additional component is 15-40% (w/w) of a lipid source.
7. The composition according to claim 6, wherein the aquaculture feed is a fish feed pellet.
8. The composition according to any of claims 1 to 7, wherein the composition further comprises one or more ingredients selected from the group consisting of fish meal, krill meal, soya concentrate, corn gluten, wheat gluten, pea protein, wheat flour, fish oil, a vitamin, mineral premix, mineral premix plus synthetic phosphorus and combinations thereof.
9. The composition according to claim 7 or 8, wherein the feed pellet is an extruded feed pellet or a pressed feed pellet.
10. The composition according to any of claims 7 to 8, wherein the feed pellet is a coated feed pellet.
11. The composition according to claim 10, wherein the active ingredient is present in the coating of the coated feed pellet.
12. The composition according to claim 11, wherein the coating comprises a coating oil and the active ingredient.
13. The composition according to claim 4 or 13, wherein said coating oil is selected from the group consisting of fish oil, microbial oil and/or one or more vegetable oil(s).
14. The composition according to claim 4, 12 or 13, wherein said coating oil comprises oleic acid (18:1n−9) in the range 0.28-229.15 g/kg feed, linoleic acid in the range 0.22-233.24 g/kg feed, alfa-linolenic acid in the range 0.28-225.06 g/kg, arachidonic acid (ARA, 20:4 n-6) in the range 0.03-24.55 g/kg, eicosapentaenoic acid (EPA, 20:5 n-3) in the range 0.03-73.66 g/kg and docosahexaenoic acid (DHA, 22:6 n-3) in the range 0.03-73.66 g/kg.
15. The composition according to claim 13, wherein the vegetable oil is selected from the group consisting of rape seed oil, soy oil and camelina oil.
16. The composition according to any of claim 4 or 12 to 15, wherein the coating oil is a source of eicosapentaenoic acid (“EPA”) and/or docosahexaenoic acid (“DHA”).
17. A method of providing an extruded feed pellet comprising as an active ingredient 25-hydroxyvitamin D, said method comprising the steps of:
a) grinding and/or mixing of at least a carbohydrate source, a protein source, a lipid source, ash, water and optionally one more ingredients selected from the group consisting of fish meal, krill meal, soya concentrate, corn gluten, wheat gluten, pea protein, wheat flour, fish oil, a vitamin, mineral premix, mineral premix plus synthetic phosphorus and combinations thereof into a mash,
b) homogenizing the mixture in (a) until a paste is formed;
c) extruding the paste obtained in step (b) by an extrusion installation comprising a mold and a number of mixing and kneading zones, composed of a plurality of alternately forward and backward kneading screw elements;
d) cutting of the extruded material into porous pellets of a suitable length when it exits the die;
e) drying of the porous pellets; and optionally, adding the steps of:
f) adding a coating oil to the pellets obtained in step (e) and adsorbing said oil into the porous pellet under vacuum, and
g) cooling of the pellets, and
h) obtaining a coated fish feed pellet.
18. The method according to claim 17, wherein the 25-hydroxyvitamin D is added in step a).
19. The method according to claim 17, wherein a composition with a coating oil according to any of claim 4 is added in step f).
20. The method according to any of claims 17 to 19, wherein the 25-hydroxyvitamin D is 25-hydroxy vitamin D3 (25-OH D3).
21. Use of 25-hydroxyvitamin D, preferably 25-hydroxyvitamin D3 (25-OH D3), for improving the feed conversion ratio (FCR), Immunity, feed efficiency (FE) protein efficiency ratio (PER) and/or the growth performance (nutrient utilization, body protein content, specific growth rate SGR, Final body weight and/or carcass yields) in aquatic animals said use comprises feeding to the aquatic animal a feed comprising 25-hydroxyvitamin D (“25-OH D3” and/or “25-OH D2”).
22. Use of 25-hydroxyvitamin D, preferably 25-hydroxyvitamin D3 (25-OH D3), for increasing levels of Vitamin D, preferably Vitamin D3, in the blood of aquatic animals said use comprises feeding to the aquatic animal a feed comprising 25-hydroxyvitamin D (“25-OH D3” and/or “25-OH D2”) and Vitamin D3.
23. The use of claim 21, wherein the animal is Vitamin D, preferably Vitamin D3, replete.
24. A method for improving the feed conversion ratio (FCR), Immunity, feed efficiency (FE) protein efficiency ratio (PER) and/or the growth performance (nutrient utilization, body protein content, protein efficiency ratio (PER) and/or the growth performance (specific growth rate SGR, Final body weight and/or carcass yields) in aquatic animals, said method comprises feeding to the aquatic animal a feed comprising 25-hydroxyvitamin D (“25-OH D3” and/or “25-OH D2”).
25. A method for increasing levels of Vitamin D, preferably Vitamin D3, in the blood of aquatic animals, said method comprises feeding to the aquatic animal a feed comprising 25-hydroxyvitamin D (“25-OH D3” and/or “25-OH D2”).
26. The method of claim 24, wherein the animal is Vitamin D, preferably Vitamin D3, replete.
27. A composition comprising 25-hydroxyvitamin D, preferably 25-hydroxyvitamin D3 (25-OH D3), for use in improving the feed conversion ratio (FCR), Immunity, feed efficiency (FE) protein efficiency ratio (PER) and/or the growth performance (nutrient utilization, body protein content, protein efficiency ratio (PER) and/or the growth performance (specific growth rate SGR, Final body weight and/or carcass yields) in aquatic animals.
28. A composition comprising 25-hydroxyvitamin D, preferably 25-hydroxyvitamin D3 (25-OH D3), for use in increasing levels of Vitamin D, preferably Vitamin D3, in the blood of aquatic animals.
29. The composition of claim 24, wherein the animal is Vitamin D, preferably Vitamin D3, replete.
30. The composition according to claim 23, wherein said composition comprises 25-hydroxyvitamin D, preferably 25-hydroxyvitamin D3 (25-OH D3), in an amount of 50 μg to 1500 μg per kg feed, preferably 100 μg to 1500 μg per kg feed, preferably 150 μg to 1500 μg per kg feed, more preferably 150 μg to 1250 μg per kg feed, more preferably 150 μg to 1000 μg per kg feed.
31. The composition according to any of claims 27 to 30, wherein said composition is administered to an aquatic animal of the group consisting of salmon, trout, bream, bass and decapod crustacean.

### EXAMPLES

### Example 1: Preparation of Pressed Fish Feed

The main raw materials are ground and mixed. Microingredients are then added to the mixer and the homogenous mix is conditioned by adding water and steam to the mass in a preconditioner. This starts a cooking process in the starch fraction (the binding component). The mass is fed into a pellet mill. The mass is forced through the mill's die and the strings are broken into pellets on the outside of the die. The moisture content is low and drying of the feed is not necessary.

The composition according to the present invention may be mixed with the main raw materials microingredients before conditioning.

Alternatively, additional oil including a fish feed composition according to the present invention is sprayed onto the surface of the pellets, but as the pellets are rather compact, the total lipid content rarely exceeds 24%. The added oil may be fish oil, microbial/algal or vegetable oils, for example rape seed oil or soy oil, or a mixture of oils. After oil coating, the pellets are cooled in a cooler and bagged.

### Example 2: Method for Preparation of Extruded Fish Feed

The main raw materials are ground and mixed. Micro ingredients incl. a fish feed composition according to the invention are added to the mixer. The homogenous mix is conditioned by adding water and steam to the mass in a preconditioner. Additional oil may also be added to the mass at this stage. This starts a cooking process in the starch fraction (the binding component). The mass is fed into an extruder. The extruder may be of the single screw or the twin-screw type. Due to the rotational movement of the mass in the extruder, the mass is further mixed. Additional oil, water and steam may be added to the mass in the extruder. At the end of the extruder, the mass has a temperature above 100° C. and a pressure above ambient pressure. The mass is forced through the openings in the extruder's die plate. Due to the relief in temperature and pressure, some of the moisture will evaporate immediately (flash off) and the extruded mass becomes porous. The strings are cut into pellets by a rotating knife. The water content is rather high (18-28%) and the pellets are therefore immediately dried to approximately 10% water content in a dryer.

The composition according to the present invention may be mixed with the main raw materials microingredients before conditioning.

Alternatively, after the dryer, more oil including a feed additive composition according to the invention may be added to the feed by spraying oil onto the surface of the feed, or by dipping the feed in oil. It is advantageous to add the oil to the feed in a closed vessel where the air pressure is below ambient (vacuum coating) so that the porous feed pellets absorb more oil. Feed containing more than 40% lipid may be produced this way. After the coater, the feed is cooled and bagged. Oil may be added at several places in the process as explained above, and may be fish oil, microbial/algal or vegetable oils, by example rape seed oil or soy oil, or a mixture of oils.

Fish need protein, fat, minerals and vitamins in order to grow and to be in good health. The diet of carnivorous fish is particularly important. Originally in the farming of carnivorous fish, whole fish or ground fish were used to meet the nutritional requirements of the farmed fish. Ground fish mixed with dry raw materials of various kinds, such as fish meal and starch, was termed soft or semi-moist feed. As farming became industrialized, soft or semi-moist feed was replaced by pressed dry feed. This was itself gradually replaced by extruded dry feed.

Today, extruded feed is nearly universal in the farming of a number of fish species such as various types of salmonid, cod, sea bass and sea bream.

The dominant protein source in dry feed for fish has been fish meal of different qualities. Other animal protein sources are also used for dry fish feed. Thus, it is known to use blood meal, bone meal, feather meal and other types of meal produced from other slaughterhouse waste, for example chicken meal. These are typically cheaper than fish meal and fish oil. However, in some geographic regions, there has been a prohibition against using such raw materials in the production of feeds for food-producing animals and fish.

It is also known to use vegetable protein such as wheat gluten, maize (corn) gluten, soya protein, lupin meal, pea meal, bean meal, rape meal, sunflower meal and rice flour.

### Example 3-Study 1: Rainbow Trout

The first study evaluated dietary 25-hydroxyvitamin D3 (25-OH D3) compared to control basal diet fed fish. Fish with a mean starting weight of 58 g were fed diets based on the same standard composition representative of typical commercial salmonid feeds.

Test diets contained no supplemented 25-OH-D₃ or either 80, 800 or 8000 μg/kg supplemented 25-OH-D₃ (as fed basis). Hy·D (25-OH-D₃) was supplemented on top of diets already containing cholecalciferol (D₃) from both endogenous and supplemented sources. The measured levels of D₃ in all experimental diets were in excess of established minimum dietary requirements for D₃, (National Research Council (NRC), Nutrient Requirements of Fish and Shrimp. 2011, Washington, DC: The National Academies Press. 392.). In addition, the dietary level of D₃ in all experimental diets met recommendations for optimal vitamin D₃ in salmonid diets (Fish Nutrition. Fourth Edition 2021. Edited by Ronald W. Hardy and Sadasivam J. Kaushik. Academic Press, UK. 905 pp.), which stipulates a vitamin D₃ supplementation of 2500-3500 international units (IU)/62.5-87.5 μg/kg). Diets were fed for 91 days following which, test fish reached a mean final weight of 332 g. Forty fish were housed in each tank and each treatment consisted of four replicate tanks.

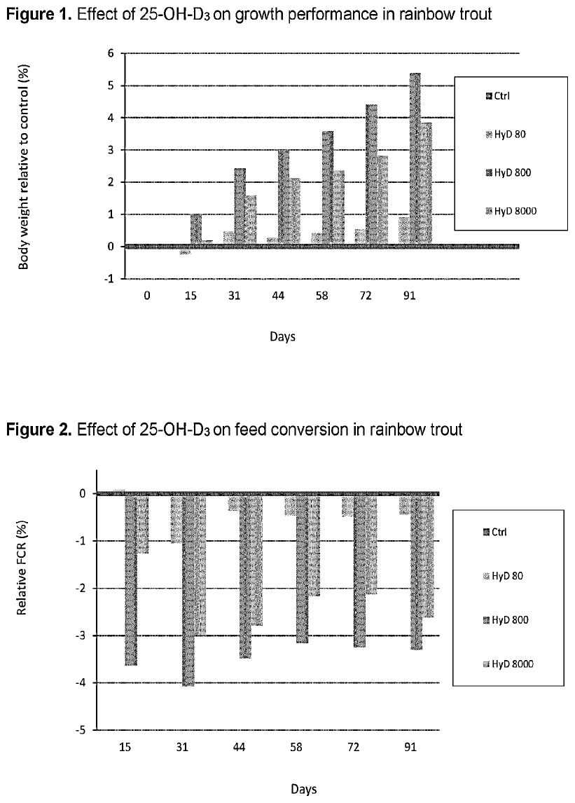

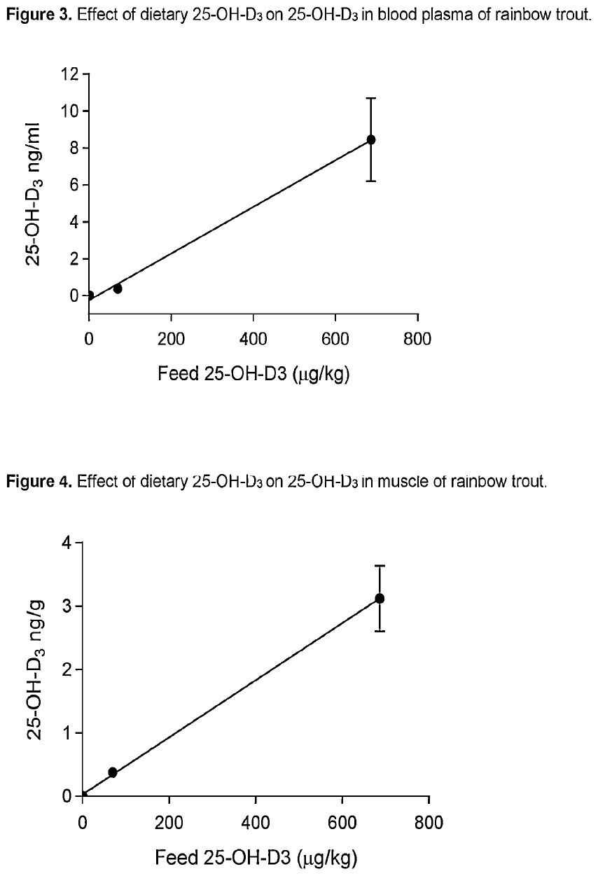

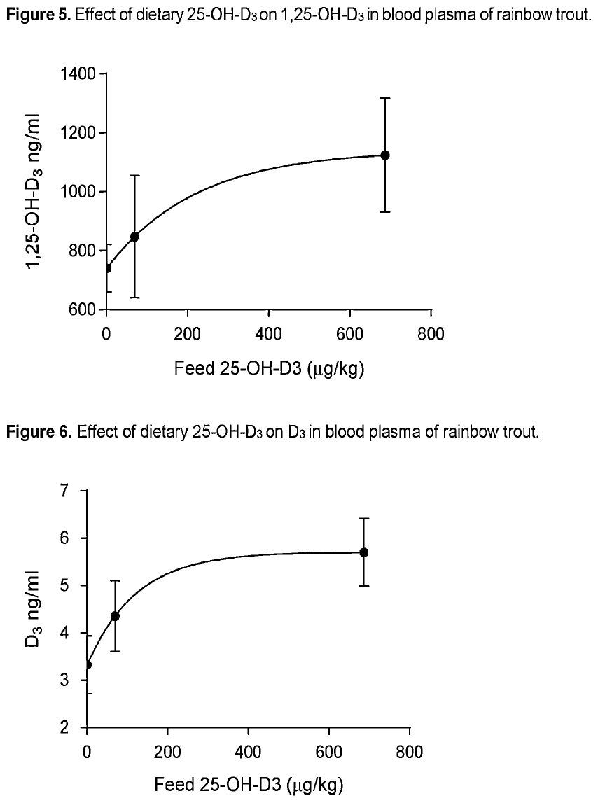

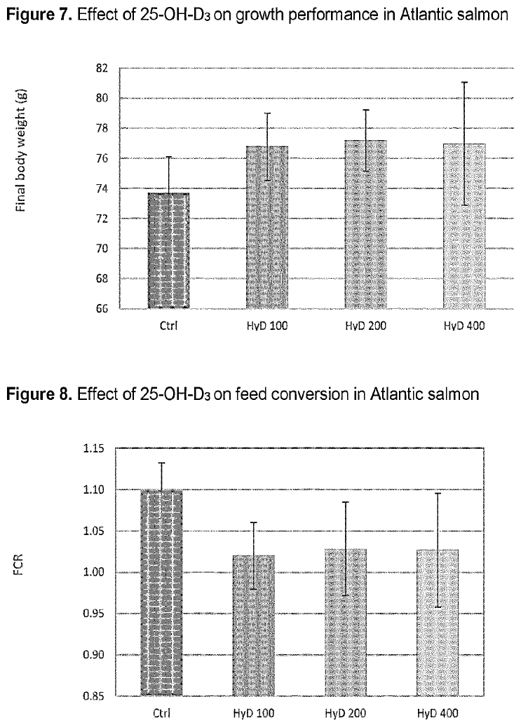

Growth performance (SGR) and feed conversion (FCR) were rapidly improved by the supplementation of 25-OH-D₃, see FIGS. 1 and 2. An assessment of changes in plasma and muscle vitamin D₃ metabolites show that 25-OH-D₃ from Hy·D is highly bioavailable to fish. Plasma and muscle 25-OH-D₃ increase in a linear relationship with supplemented dietary 25-OH-D₃, see FIGS. 7 and 8. Despite diets containing recommended levels of D₃, Hy·D is required to effectively raises the status of both plasma vitamin D₃ and 1,25 dihydroxycholecalciferol, the active form of vitamin D₃, see FIGS. 5 and 6.

### Example 4—Study 2: A. salmon

The second study evaluated dietary 25-hydroxyvitamin D₃ (25-OH D₃) compared to control basal diet fed fish.

Fish with a mean starting weight of 23.4 g were fed diets based on the same standard composition representative of typical commercial salmonid feeds. Test diets contained no supplemented 25-OH-D₃ or either 100, 200 or 400 μg/kg supplemented 25-OH-D₃ (as fed basis). Hy·D (25-OH-D₃) was supplemented on top of diets already containing cholecalciferol (D₃) from both endogenous and supplemented sources. The measured levels of D₃ in all experimental diets were in excess of established minimum dietary requirements for D₃, (National Research Council (NRC), Nutrient Requirements of Fish and Shrimp. 2011, Washington, DC: The National Academies Press. 392.). In addition, the dietary level of D₃ in all experimental diets met recommendations for optimal vitamin D₃ in salmonid diets (Fish Nutrition. Fourth Edition 2021. Edited by Ronald W. Hardy and Sadasivam J. Kaushik. Academic Press, UK. 905 pp.), which stipulates a vitamin D₃ supplementation of 2500-3500 international units (IU)/62.5-87.5 μg/kg). Diets were fed for 90 days with test fish reaching a mean final weight of 73.0 g. Twenty fish were housed in each tank and each treatment consisted of five replicate tanks.

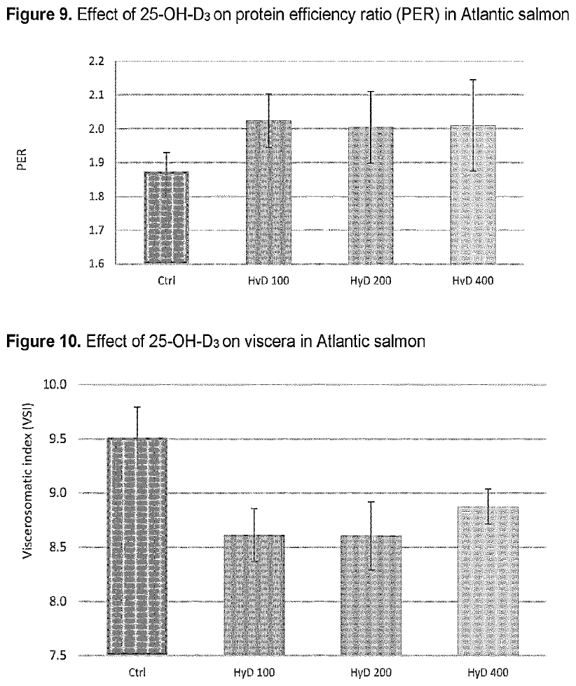

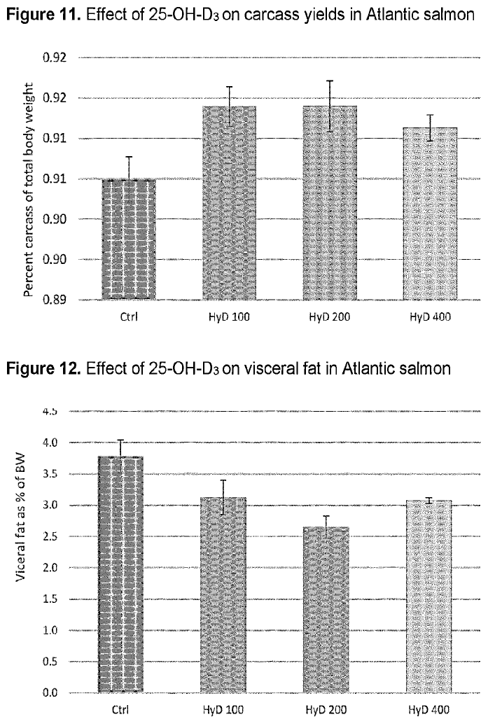

Final body weight, feed conversion (FCR) and protein efficiency ratio (PER) were all improved by the supplementation of 25-OH-D₃ from Hy·D, see FIGS. 7, 8 and 9. The supplementation of 25-OH-D₃ from Hy·D also improved carcass yields at the expense of peritoneal fat surrounding the viscera, see FIGS. 10, 11 and 12. The decrease in visceral fat relative to the carcass (whole body weight minus viscera) is shown by the reduced VSI in all fish supplemented 25-OH-D₃ from Hy·D. This results in a greater carcass yield relative to the whole fish, as shown in FIG. 11. The reduced VSI is confirmed to be due to a reduced percentage fat being deposited in the viscera, see FIG. 12. Collectively, these data show that supplementation of 25-OH-D₃ from Hy·D results a reduced fat deposition in the viscera with a concurrent increase in carcass yields.

### Example 5-Study 1: Rainbow Trout

The first study evaluated standard dietary levels of supplemented vitamin D3 alone or in combination with 80 μg/Kg 25-hydroxy vitamin D3dietary 25-hydroxyvitamin D3 (25-OH D3) in rainbow trout fed for 20 weeks from first-feeding.

Two commercial-like experimental diets were formulated by DSM Nutritional Products France and produced by SPAROS (Portugal). The diet compositions are shown in the Table 1. The basal diet was supplemented with either vitamin D3 only (VitD3 diet) or D3 in combination with 80 μg/Kg 25-hydroxy vitamin D3 (25-OH-D3 diet) (Table 1).

TABLE 1
Compositions of the experimental diets.

| | Pellets 1-8 weeks | | Pellets 8-20 weeks | |
|---|---|---|---|---|
| | (200-1000 microns) | | (1.5-2-3 mm) | |
| | | 25-OH-D3 | | 25-OH-D3 |
| | VitD3 Diet | Diet | VitD3 Diet | Diet |
| Ingredients. % | | | | |
| Fishmeal LT70 | 45.000 | 45.000 | 30.000 | 30.000 |
| Fish protein concentrate | 15.000 | 15.000 | 5.000 | 5.000 |
| Fish gelatin | 1.000 | 1.000 | | |
| Soy protein concentrate | | | 13.500 | 13.500 |
| Pea protein concentrate | 6.000 | 6.000 | 6.000 | 6.000 |
| Wheat gluten | 12.500 | 12.500 | 12.500 | 12.500 |
| Wheat meal | 6.400 | 6.400 | 15.180 | 15.180 |
| Fish oil | 6.000 | 6.000 | 6.000 | 6.000 |
| Linseed oil | 0.800 | 0.800 | 0.800 | 0.800 |
| Rapeseed oil | | | 9.000 | 9.000 |
| Soy lecithin | 6.280 | 6.280 | 1.000 | 1.000 |
| DSM VMP1 - 0.5% | 0.500 | 0.500 | 0.500 | 0.500 |
| 25(OH)D3 | | 0.008 | | 0.008 |
| Vitamin E (ROVIMIX E50) | 0.020 | 0.020 | | |
| Sodium phosphate (NaH2PO4) | 0.500 | 0.500 | 0.500 | 0.500 |
| Lutavit E50 | | | 0.020 | 0.020 |
| Analytical composition | | | | |
| Dry Matter ((DM) | 93.26 | 93.34 | 93.28 | 95.09 |
| Protein (% DM) | 63.16 | 63.40 | 54.25 | 54.30 |
| Lipids (% DM) | 18.79 | 18.44 | 20.57 | 20.59 |
| Starch (% DM) | 6.73 | 6.76 | 14.54 | 14.31 |
| Energy | 23.20 | 23.07 | 23.97 | 24.06 |
| Ash (% DM) | 10.86 | 10.82 | 8.10 | 8.16 |

The parameters for growth performance, including feed efficiency (FE), were measured every 3 weeks and calculated as follow: Feed efficiency (FE)=(the mass for dead fish+final wet body mass-initial wet body mass)/dry feed intake. Where d is the experimental period in days.

Vitamin D and its metabolites (25-OH-D3, 3-epi-25-OH-D3, 24-(R),25-dyhidoryvitamin D₃ and 25-hydroxivitamin D₂) were determined in plasma samples obtained after 20 weeks of experimental feeding using UPLC (1290 Agilent Infinity II LC System) coupled with MS detection (API4000 SCIEX) by the R&D Solution Centre, DSM Nutritional products, Kaiseraugst, Switzerland. Briefly, following protein precipitation and extraction vitamin D metabolites were analyzed using a LC-MS/MS system. To ensure analytical accuracy, daily and long-term laboratory checks on the method performance were performed using dedicated standards and quality-control samples. These were analyzed against unknown samples to validate the accuracy and precision of the method. Data acquisition, integration and quantification were performed by Analyst® software (Sciex).

Plasma glucose (Glucose RTU. bioMérieux, Marcy l'Etoile. France), NEFA C (Fuji Chemicals GmbH) and triglycerides (Triglycerides PAP 150. bioMérieux) levels were determined using commercial kits adapted to a microplate format, according to the recommendations of the manufacturer. Total plasma free amino acid concentrations were determined by the ninhydrin reaction according to the method of Moore (1951) with glycine as standard.

Gene Expression Analysis Through qPCR

For each sampling, gene expression for some key actors of the intermediary metabolism were studied in liver by qRT-PCR. At least 40 genes were determined based on their key enzymatic roles in lipid metabolism β-oxydation), mitochondrial energy metabolism (enzymes of the Krebs cycle), glucose metabolism (glycolytic and gluconeogenic enzymes) and protein metabolism (amino acid catabolism). We also analysed the glucose transporters gluts (2 glut2, 2 glut4 and 2 glut1). The primer sequences used in the real-time RT-PCR assays, as well as the protocol conditions of the assays for mRNAs of glucose, lipid, energy and amino acid metabolic genes have been previously published in different studies (Song X, Marandel L, Dupont-Nivet M, Quillet E, Geurden I, Panserat S. 2018a. and Song X, Marandel L, SkibaCassy S, Corraze G, Dupont-Nivet M, Quillet E, Geurden I, Panserat S. 2018b.).

Total RNA was extracted from liver (n=9 samples per dietary treatment). Samples were homogenized using Precellys® 24 (Bertin Technologies, Montigny-le-Bretonneux, France) in tubes containing Trizol reagent (Invitrogen, Carlsbad, CA, USA). Total RNA was then extracted according to the manufacturer's instructions. The total RNA concentrations were estimated spectrophotometrically using a NanoDrop 2000. The quality of extracted RNA was determined after migration on a 1% agarose by gel electrophoresis. Total RNA (1 μg) was used for cDNA synthesis by reverse transcription. The Super-Script III RNAse H-Reverse transcriptase kit (Invitrogen, Carlsbad, California, USA) was used with random primers (Promega) to synthesize cDNA. For real-time RT-PCR assays of transcripts of metabolic genes, the Roche Lightcycler 480 system was used (Roche Diagnostics. Neuilly-sur-seine. France). The assays were performed using a reaction mix of 6 μl per sample, each of which contained 2 μl of diluted cDNA template, 0.12 μl of each primer (10 μM), 3 μl Light Cycler 480 SYBR® Green I Master mix and 0.76 μl D and bNAse/RNAse free water (5 Prime GmbH. Hamburg. Germany). The PCR protocol was initiated at 95° C. for 10 min for initial denaturation of the cDNA and hot-start Taq-polymerase activation, followed by 45 cycles of a two-step amplification programme (15 s at 95° C.; 40 s at 60-64° C.) according to the primer set used. Melting curves were systematically monitored (temperature gradient at 1.1° C./10 s from 65-94° C.) at the end of the last amplification cycle to confirm the specificity of the amplification reaction. Each PCR assay included replicate samples (duplicate of reverse transcription and PCR amplification, respectively) and negative controls (reverse transcriptase- and cDNA template-free samples, respectively). For the expression analysis of mRNA, relative quantification of target gene expression was performed using the ΔCT method (Pfaffl. 2001). The relative gene expression of EF1A were used for the normalization of measured mRNAs in alevins and juveniles as its relative expression did not significantly change over sampling time. In all cases, PCR efficiency (E) was measured by the slope of a standard curve using serial dilutions of cDNA. In all cases, PCR efficiency values ranged between 1.8 and 2.2

### Transcriptomics Analysis

Gene expression was evaluated by One-color microarray-based analysis using a custom Rainbow trout Gene Expression Microarray, 4×180K (Agilent: G4862A-ID.086588). 100 ng of total RNA (n=6 per treatment) was labelled with Low Input Quick Amp Labelling Kit following manufacturer's instructions. The quality of the Cy-3 labelled cRNA was checked using a NanoDrop and the yield and specific activity were calculated. Hybridization and scanning were done following the protocol described by Agilent. Signal intensities obtained were extracted using Feature Extraction software version 12.1. The extracted data were analyzed using Partek® Genomics Suite® version 7 to identify the differentially expressed genes. The pathway analysis was performed on the IPA software against human pathway database based on orthologous genes alignment. Gene ontology analysis was performed using String software.

### Statistical Analysis

Data are presented as means±standard deviation (SD). For growth parameters, the plasma and the qPCR results, data were analyzed by t-Student comparison of two means. Normality was assessed using the Shaprio-Wilk test, while homoscedasticity was determined using Levene's test. Data was analysed using the statistical R software/R Commander package. For all statistical analyses, the level of significance was set at P<0.05. For transcriptomics studies, statistical analyses were performed on raw data gene expression measurement using one-way ANOVA and Tukey as post-hoc test. Differentially expressed genes (DEG) are determined and hierarchical clustering is illustrated by a heatmap. A cut-off of fold changes was set at >1.5 or <−1.5 and unadjusted p-value at 0.05, comparing treatment VitD3 Diet versus 25-OH-D3 Diet.

After 20 weeks of feeding, fry fish fed the dietary 25-hydroxy Vitamin D3 showed a significant increase in feed efficiency (1.29 vs 1.19; p=0.0002) indicating a improves nutrient utilisation. The whole body composition of fish after 20 weeks of feeding showed that fish fed with 25-OH-D3 had a higher whole body protein (47.38 vs. 51.15; p=0.006).

Surprisingly 25-hydroxy Vitamin D3 has an apparent impact on the transcriptome profile in the liver. 1301 genes were found to be differentially expressed (DEG) between the 25-OH-D3 supplemented group and the control group, showing effects of 25-OH-D3 on liver metabolism of rainbow trout. Our data show the modulation of several pathways involved in fish immunity. For example, modulation of the pattern recognition receptors in recognition of bacteria and viruses and LPS/IL-1 mediated inhibition of RXR function are clearly linked to immune response. In addition, modulation of Role of RIG1-like Receptors in Antiviral Innate Immunity pathway, which are key sensors of virus infection, mediating the transcriptional induction of type I interferons and other genes that collectively establish an antiviral host response. These gene modulations described, indicate that 25-hydroxy vitamin D3 supplementation in rainbow trout supporting immune function.

Based on our data, the involvement of 25-hydroxy vitamin D3 in innate immunity is confirmed by modulation of Aldosterone Signaling in Epithelial Cells pathway, coupled with modulation of Production of Nitric Oxide and Reactive Oxygen Species in Macrophages pathway.

In our study, DNAJC28 also known as Heat Shock Protein 40, HSPA8 also known as Heat Shock Protein 70 and HSPB1 also known as Heat Shock Protein 27 are all upregulated with HyD supplementation. These molecules are known to play cytoprotective activities under pathological conditions, as for example oxidative stress. Also, the analysis of Production of Nitric Oxide and Reactive Oxygen Species in Macrophages pathway shows a decrease of nitric oxide and anion superoxide generation by modulation of some transcription factors. These observations suggest that HyD supplementation play a key role in innate immunity in rainbow trout.

TABLE 2
Upregulated pathways. Enrichment analysis has been made using String Software version 11.5.

| | | % genes in | | |
|---|---|---|---|---|
| Ingenuity Canonical | Enrichment | pathway that | | |
| Pathways | p-value | are present | z-score | Genes involved in the pathway |
| Aldosterone Signaling | 0.0219 | 10.30 | −1.633 | DNAJC28, HSPA8, HSPB1, |
| in Epithelial Cells | | | | ICMT, NR3C2, PDIA3, |
| | | | | PIK3R1, PLCE1, PRKCA |
| LPS/IL-1 Mediated | 0.0288 | 8.33 | 1.134 | ACSL1, ACSL3, ALDH1L2, CPT2, |
| Inhibition of RXR | | | | CYP2C18, CYP7A1, FABP1, |
| Function | | | | MAOA, NGFR, NROB2, PAPSS2, |
| | | | | PPARGC1A, SCARB1, SOD3 |
| Role of RIG1-like | 0.0068 | 19.20 | 1 | CASP8, MAVS, RELB, |
| Receptors in Antiviral | | | | TRAF3, TRIM25 |
| Innate Immunity | | | | |
| Production of Nitric | 0.0646 * | 8.20 | 0.71 | APOA4, APOF, FOS, NGFR, |
| Oxide and Reactive Oxygen | | | | PIK3R1, PPP1R14B, PPP2CA, |
| Species in Macrophages * | | | | PRKCA, RELB, SPI1 |

1. A composition comprising as an active ingredient 25-hydroxyvitamin D, wherein the composition is intended for intake by aquatic animals and wherein and the concentration of the active ingredient in the final feed added to the animal is in the range from 50 μg to 1500 μg per kg feed, preferably 100 μg to 1500 μg per kg feed, preferably 150 μg to 1500 μg per kg feed, more preferably 150 μg to 1250 μg per kg feed, more preferably 150 μg to 1000 μg per kg feed.

2. The composition according to claim 1, wherein the 25-hydroxyvitamin D is 25-hydroxy vitamin D3 (25-OH D3).

3. The composition according to claim 1, wherein the active ingredient is embedded in an oil carrier.

4. The composition according to claim 1, wherein said composition is a feed premix or an aquaculture feed.

5. The composition according to claim 4, wherein the aquaculture feed is a coated feed pellet.

6. The composition according to claim 5, wherein the active ingredient is present in the coating of the coated feed pellet.

7. The composition according to claim 6, wherein the coating comprises a coating oil and the active ingredient.

8. A method of providing an extruded feed pellet comprising as an active ingredient 25-hydroxyvitamin D, said method comprising the steps of:
a) grinding and/or mixing of at least a carbohydrate source, a protein source, a lipid source, ash, water and optionally one more ingredients selected from the group consisting of fish meal, krill meal, soya concentrate, corn gluten, wheat gluten, pea protein, wheat flour, fish oil, a vitamin, mineral premix, mineral premix plus synthetic phosphorus and combinations thereof into a mash,
b) homogenizing the mixture in (a) until a paste is formed;
c) extruding the paste obtained in step (b) by an extrusion installation comprising a mold and a number of mixing and kneading zones, composed of a plurality of alternately forward and backward kneading screw elements;
d) cutting of the extruded material into porous pellets of a suitable length when it exits the die;
e) drying of the porous pellets; and optionally, adding the steps of:
f) adding a coating oil to the pellets obtained in step (e) and adsorbing said oil into the porous pellet under vacuum, and
g) cooling of the pellets, and
h) obtaining a coated fish feed pellet.

9. The method according to claim 8, wherein the 25-hydroxyvitamin D is added in step a).

10. The method according to claim 8, wherein a composition with a coating oil according to any of claim 4 is added in step f).

11. The method according to claim 8, wherein the 25-hydroxyvitamin D is 25-hydroxy vitamin D3 (25-OH D3).

12. Use of 25-hydroxyvitamin D, preferably 25-hydroxyvitamin D3 (25-OH D3), for improving the feed conversion ratio (FCR), Immunity, feed efficiency (FE) protein efficiency ratio (PER) and/or the growth performance (nutrient utilization, body protein content, protein efficiency ratio (PER) and/or the growth performance (specific growth rate SGR, Final body weight and/or carcass yields) in aquatic animals said use comprises feeding to the aquatic animal a feed comprising 25-hydroxyvitamin D (“25-OH D3” and/or “25-OH D2”).

13. Use of 25-hydroxyvitamin D, preferably 25-hydroxyvitamin D3 (25-OH D3), for increasing levels of Vitamin D, preferably Vitamin D3, in the blood of aquatic animals said use comprises feeding to the aquatic animal a feed comprising 25-hydroxyvitamin D (“25-OH D3” and/or “25-OH D2”) and Vitamin D3.

14. The use of claim 13, wherein the animal is Vitamin D, preferably Vitamin D3, replete.

15. A composition comprising 25-hydroxyvitamin D, preferably 25-hydroxyvitamin D3 (25-OH D3), for use in improving the feed conversion ratio (FCR), Immunity, feed efficiency (FE) protein efficiency ratio (PER) and/or the growth performance (nutrient utilization, body protein content, protein efficiency ratio (PER) and/or the growth performance (specific growth rate SGR, Final body weight and/or carcass yields) in aquatic animals.

16. A composition comprising 25-hydroxyvitamin D, preferably 25-hydroxyvitamin D3 (25-OH D3), for use in increasing levels of Vitamin D, preferably Vitamin D3, in the blood of aquatic animals.

17. The composition of claim 16, wherein the animal is Vitamin D, preferably Vitamin D3, replete.

18. The composition according to claim 15, wherein said composition comprises 25-hydroxyvitamin D, preferably 25-hydroxyvitamin D3 (25-OH D3), in an amount of 50 μg to 1500 μg per kg feed, preferably 100 μg to 1500 μg per kg feed, preferably 150 μg to 1500 μg per kg feed, more preferably 150 μg to 1250 μg per kg feed, more preferably 150 μg to 1000 μg per kg feed.
